(19) United States
(12) Patent Application Publication
(10) Pub. No.: US 2021/0000889 A1
(43) Pub. Date: Jan. 7, 2021

(54) METHODS FOR PROGNOSING AND TREATING METABOLIC DISEASES

(71) Applicants: INSERM (INSTITUT NATIONAL DE LA SANTÉ ET DE LA RECHERCHE MÉDICALE), Paris (FR); UNIVERSITÉ DE PARIS, Paris (FR); SORBONNE UNIVERSITÉ, Paris (FR); ASSISTANCE PUBLIQUE-HÔPITAUS DE PARIS (APHP), Paris (FR); CENTRE NATIONAL DE LA RECHERCHE SCIENTIFIQUE (CNRS), Paris (FR)

(72) Inventors: Fatima-Soraya TALEB, Paris (FR); Harry SOKOL, Paris (FR); Ludivine LAURANS, Paris (FR)

(21) Appl. No.: 16/977,274

(22) PCT Filed: Feb. 28, 2019
(86) PCT No.: PCT/EP2019/054946

§ 371 Date: Sep. 1, 2020

(30) Foreign Application Priority Data
Mar. 1, 2018 (EP) 18305219.0
Jul. 16, 2018 (EP) 18305960.9

(51) Int. Cl. A61K 35/741; A23L 33/135; A61P 3/04; A23L 33/00

(52) U.S. Cl. CPC A61K 35/741; A23L 33/135; A61K 45/06; A23L 33/30; A61P 3/04

(57) ABSTRACT

The present invention relates to methods for prognosing and treating metabolic diseases. The inventors demonstrated the association of obesity with the increase of intestinal IDO activity, which shifts tryptophan (Trp) metabolism from indole derivative but also IL-22 production towards kynurenine (Kyn) production. The inventors showed that the rewiring of Tip metabolism is possible towards a microbiota-dependent production of IL-22. In particular, the present invention relates to a method of treating metabolic diseases in a subject in need thereof comprising administering to the subject a therapeutically effective amount of probiotics

### FIELD OF THE INVENTION

The present invention relates to methods for prognosing and treating metabolic diseases.

### BACKGROUND OF THE INVENTION

Metabolic diseases are a major health concern and typically include disorders such as obesity, diabetes or hypertension. The association between an altered gut microbiota, intestinal permeability and metabolic disorders, is becoming increasingly clear but remains poorly understood.

Obesity is a condition characterized by an excess of body fat. The prevalence of overweight and obesity is considered an important public health issue in the world. Roughly two thirds of US adults meet the criteria for overweight or obesity. Actually, obesity is an important risk factor for coronary heart disease (CHD), ventricular dysfunction, congestive heart failure, stroke, and cardiac arrhythmias. Furthermore obesity is closely associated with type 2 diabetes, metabolic syndrome and hepatic disorders such as non-alcoholic fatty liver disease. Furthermore epidemiologic evidences suggest that obesity increases the risk of cirrhosis. Weight loss drugs that are currently used for the treatment of obesity have limited efficacy and significant side effects. However the side effects of current drugs limit their use.

For instance dexfenfluramine was withdrawn from the market because of suspected heart valvulopathy.

Thus there still is a need for treating and prognosing metabolic disorders and in particular obesity.

### SUMMARY OF THE INVENTION

The present invention relates to methods for prognosing and treating metabolic diseases. In particular, the present invention is defined by the claims.

### DETAILED DESCRIPTION OF THE INVENTION

The inventors previously showed that obesity is associated with an increase of intestinal indoleamine 2-3 dioxygenase (IDO) activity, which shifts tryptophan (Trp) metabolism. They showed the beneficial effect of IDO invalidation on body weight and fat mass, insulin sensitivity and inflammation.

Here the inventors demonstrate more precisely the association of obesity with the increase of intestinal IDO activity, which shifts Trp metabolism from indole derivative but also IL-22 production towards kynurenine (Kyn) production. The inventors demonstrate that the beneficial effects previously showed are due to rewiring of Trp metabolism towards a microbiota-dependent production of IL-22.

More particularly, the inventors show differences in the microbiota composition: lower proportions of Bacteroidetes phylum and especially Rikenellaceae family are observed in obese mice.

Moreover, the inventors show for the first time that kyn levels in feces are higher in obese mice.

Accordingly, a first object of the present invention relates to a method of treating metabolic diseases in a subject in need thereof comprising administering to the subject a therapeutically effective amount of probiotics.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

By a “therapeutically effective amount” is meant a sufficient amount of the probiotics of the present invention for reaching a therapeutic effect. It will be understood, however, that the total daily usage of the probiotics and compositions containing probiotics of the present invention will be decided by the attending physician within the scope of sound medical judgment. The specific therapeutically effective dose level for any particular subject will depend upon a variety of factors including the disorder being treated and the severity of the disorder; activity of the specific probiotics employed; the specific composition employed, the age, body weight, general health, sex and diet of the subject; the time of administration, route of administration, and rate of excretion; the duration of the treatment; drugs used in combination or coincidental with the probiotics employed; and like factors well known in the medical arts. For example, it is well within the skill of the art to start doses of the probiotics at levels lower than those required to achieve the desired therapeutic effect and to gradually increase the dosage until the desired effect is achieved.

As used herein, the term “subject” denotes a mammal, such as a rodent, a feline, a canine, and a primate. Preferably, a subject according to the invention is a human.

As used herein, the term “metabolic disease” denotes a disease that disrupts normal metabolism. In a preferred embodiment, the metabolic diseases is selected from the group consisting of diabetes, obesity, hypertension, elevated plasma insulin concentrations and insulin resistance, dyslipidemia, and hyperlipidemia. In a preferred embodiment, the metabolic disease is obesity.

As used herein, the term “probiotic” refers to a live microorganism which when administered in adequate therapeutic amounts confer a health benefit on a subject. Health benefits are a result of production of nutrients and/or co-factors by the probiotic, competition of the probiotic with pathogens and/or stimulation of an immune response in the subject by the probiotic.

In one embodiment, the probiotic is Bacteroidetes.

As used herein, the term “Bacteroidetes” is well-known in the art and refers to a bacteria phylum which is composed of three large classes of Gram-negative, non sporeforming, anaerobic or aerobic, and rod-shaped bacteria.

In one embodiment, the probiotic is Rikenellaceae.

As used herein, the term “Rikenellaceae” refers to a bacteria family (phylum: Bacteroidetes).

The metabolism of Rikenellaceae bacteria is anaerobic and acid is produced from glucose, lactose, mannose and melibiose. Metabolic endproducts include alcohols, acetic acid, proprionic acid and succinic acid.

A further object of the present invention relates to a method of treating metabolic diseases in a subject in need thereof comprising administering to the subject a therapeutically effective amount of probiotics wherein the probiotics is not Bacteroides uniformis strain with deposit number CECT 7771 probiotics.

A further object of the present invention relates to a method of treating metabolic diseases in a subject in need thereof comprising administering to the subject a therapeutically effective amount of probiotics wherein the probiotics is not Bacteroides uniformes probiotics.

A further object of the present invention relates to a method of treating metabolic diseases in a subject in need thereof comprising administering to the subject a therapeutically effective amount of probiotics wherein the probiotics is not Bacteroides genus probiotics.

A further object of the present invention relates to a method of treating metabolic diseases in a subject in need thereof comprising administering to the subject a therapeutically effective amount of Bacteroidetes probiotics wherein the probiotics is not Bacteroides uniformis strain with deposit number CECT 7771 probiotics.

As used herein, the term “Bacteroides” is well-known in the art and refers to the genus of Gram-negative, obligate anaerobic bacteria. As used herein, the term “Bacteroides uniformis” is well-known in the art and refers to a bacteria species which belongs to the Bacteroides genus. In a particular embodiment, the Bacteroides uniformis bacteria is Bacteroides uniformis strain with deposit number CECT 7771.

Another aspect of the present invention relates to a method of improving insulin sensitivity in a subject in need thereof comprising administering to the subject a therapeutically effective amount of probiotics. In one embodiment, the probiotic is probiotics is Bacteroidetes. In one embodiment, the probiotic is probiotics is Rikenellaceae. In one embodiment, the probiotic is not Bacteroides uniformis strain with deposit number CECT 7771 probiotic. In one embodiment, the probiotic is not Bacteroides uniformes probiotic. In one embodiment, the probiotic is not Bacteroides genus probiotic.

As used herein, the term “insulin sensitivity” refers to the ability of a cell, tissue, organ or whole body to absorb glucose in response to insulin.

As used herein, the term “improving insulin sensitivity” refers to the improvement of insulin sensitivity.

In particular, the method of the present invention is particularly suitable for controlling weight gain or for stimulating weight loss in a subject in need thereof comprising administering to the subject a therapeutically effective amount of probiotics. In one embodiment, the probiotic is probiotics is Bacteroidetes. In one embodiment, the probiotic is probiotics is Rikenellaceae. In one embodiment, the probiotic is not Bacteroides uniformis strain with deposit number CECT 7771 probiotic. In one embodiment, the probiotic is not Bacteroides uniformes probiotic. In one embodiment, the probiotic is not Bacteroides genus probiotic.

Another aspect of the present invention relates to compositions comprising probiotics of the invention.

Another aspect of the present invention relates to compositions comprising Bacteroidetes probiotics of the invention.

Another aspect of the present invention relates to compositions comprising Rikenellaceae probiotics of the invention.

According to the invention, the probiotics is administered to the subject in the form of a dietary supplement or in the form of pharmaceutical composition. Typically, the probiotics may be combined with any excipient including for example pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the case of pharmaceutical compositions for oral or rectal administration, the probiotic, alone or in combination with another active principle, can be administered in a unit administration form, as a mixture with conventional pharmaceutical supports, to animals and human beings.

Suitable unit administration forms comprise oral-route forms such as tablets, gel capsules, powders, granules and oral suspensions or solutions, sublingual and buccal administration forms, and rectal administration forms.

In order to exert these beneficial effects on the host, probiotics must retain their viability and reach the large intestine in therapeutic quantities (Favaro-Trindade, C. S., et al. (2002), J Microencapsulation 19(4): 485-494)). Effective probiotic bacteria should be able to survive gastric conditions and colonize the intestine, at least temporarily, by adhering to the intestinal epithelia (Conway, P. (1 96), Selection criteria for probiotic microorganisms. Asia Pacific J. Clin. Nutr 5: 10-14).

For example, probiotics may be added in dietary supplement forms, such as powders, capsules and tablets. Probiotics administration may require an effective delivery system that retains probio-functional activities (i.e., gutadhesion/retention, production of bacteriocins/enzymes) after their revival (Salminen, S., et al. (1996), Clinical uses of probiotics for stabilizing the gut mucosal barrier: successful strains and future challenges. Antonie Van Leeuwenhoek 70:347-3581). Furthermore, in addition to increasing in vivo viability and gastrointestinal tract life span, prolonged shelf life at room temperature remains an important factor. Though freeze-drying of the probiotic bacteria has been shown to be an effective process for preservation and delivery of probiotics, several physico-chemical factors such as humidity, aeration (oxygen availability), processing (i.e., agitation), and temperature could compromise the cell viability, shelf life and, accordingly its therapeutic use.

The stability, viability (i.e., viable microbial content) and quality of products containing probiotic bacteria are problematic. The predominant challenges to stability of probiotic bacteria are water activity, physical stress of processing and temperature. It has also been challenging to apply protective measures, such as coatings, that will release the probiotic bacteria at the appropriate delivery site in the body and allow the probiotic to colonize. The appropriate delivery and colonization of the coated probiotic bacteria has recently been confirmed in a newly published study (Del Piano, M., et al. (2010, Evaluation of the intestinal colonization by microencapsulated probiotic bacteria in comparison to the same uncoated strains, Journal of Clinical Gastroenterology, 44 Supp. 1: S42-6).

Oil suspensions have been utilized to increase the viability and shelf life of probiotics. For example, U.S. Patent Application Publication No. 2004/0223956 discloses a composition containing probiotic bacteria suspended in an edible oil and, optionally, encapsulated in a two piece hard shell capsule. In addition, those in the art have tried using probiotic microspheres to enhance viability and shelf life. For example, U.S. Patent Application Publication No. 2005/0266069 discloses probiotic formulations containing probiotic microspheres having a core of a probiotic bacteria and a cellulosic excipient coated with coating agents and plasticizers.

Experience has long shown that pharmaceuticals or other items for human or animal consumption may be safely and conveniently packaged in a hard or soft gelatin shell (softgel). Filled one-piece soft capsules or softgels have been widely known and used for many years and for a variety of purposes.

Encapsulation within a soft capsule of a solution or dispersion of a nutritional or pharmaceutical agent in a liquid carrier offers many advantages over other dosage forms, such as compressed, coated or uncoated solid tablets, or bulk liquid preparations. Encapsulation of a solution or dispersion permits accurate delivery of a unit dose. Soft capsules provide a dosage form that is easy to swallow and need not be flavored, a good oxygen barrier (i.e., low oxygen permeability through the capsule shell), and tamper protection. Soft capsules are also more easily transported than food products and liquids, such as yogurt and milk.

For example, the capsules may be admixed with oligosaccharides, sweeteners and flavors and presented in individually wrapped, single dose aluminum tubes.

In order to improve organoleptic properties of the administration form of the probiotics, any compound or substance may be added such as colouring or flavour for instance.

The dosage form must be sufficiently robust such that a sufficient number of viable probiotic bacteria survive manufacturing conditions and storage, in order to exert a beneficial effect when in use. This problem is compounded by the fact that it is particularly important to have a high viable microbial count in a unit dosage form intended to treat, because a high proportion of the probiotic bacteria can be expected to be lost to the oral cavity because of ingestion.

The count of viable probiotic bacteria obtained can be determined by standard laboratory dilution methods generally known in the art, such as plating a quantified dilution of bacteria onto agar plates and then performing a colony count.

A typical dosage form will contain about 1.0 to 10000 mg, more particularly about 100 to about 5000 mg of probiotic bacteria.

A further object of the present invention relates to a method of treating a metabolic disease in a subject in need thereof comprising administering to the subject a therapeutically effective amount of at least one ligand of aryl hydrocarbon receptor (AHR).

A further object of the present invention relates to a method of improving insulin sensitivity in a subject in need thereof comprising administering to the subject a therapeutically effective amount of at least one ligand of aryl hydrocarbon receptor (AHR).

A further object of the present invention relates to a method of controlling weight gain or of stimulating weight loss in a subject in need thereof comprising administering to the subject a therapeutically effective amount of at least one ligand of aryl hydrocarbon receptor (AHR).

As used herein, the “ayrl hydrocarbon receptor” or “AHR” has its general meaning in the art and is a ligand activated transcription factor of the basic region helix-loop-helix-PER/ARNT/SIM homology family. Accordingly, the term “ligand of AHR” refers to any compound natural or not that is capable to binding AHR and promotes activation of the signaling pathway of AHR. The prototypic signaling pathway of AHR-mediated transcriptional activity is characterized by transcription of a battery of drug-metabolizing enzymes, which includes cytochrome P450 enzymes 1A1, 1A2, and 1B1.

In some embodiments, the ligand is selected from the group consisting of 2,3,7,8-Tetrachlorodibenzo-p-dioxin (TCDD), lndole-3-carbinol (I3C), lndole-3-acetonitrile (I3ACN), 3,3-Diindolylmethane (DIM), 2-(Indol-3-ylmethyl)-3,3′-diindolylmethane (Ltr-1), Indolo [3,2-b]carbazole (ICZ), 2-(1′ H-indole-3′-carbonyl)-thiazole-4-carboxylic acid methyl ester (ITE), Indole, lndole-3-acetic acid (IAA), lndole-3-aldehyde (IAld), Tryptamine, 3-Methyl-indole (skatole), lndoxyl-3-sulfate (I3S), Kynurenine (Kyn), Kynurenic acid (KA), Xanthurenic acid, Cinnabarinic acid (CA), and 6-Formylindolo[3,2-b]carbazole (FICZ).

Typically the ligand of AHR is administered to the subject in a food composition (for oral administration). In some embodiments, the ligand of AHR is administered to the subject in a form of a pharmaceutical composition.

The invention will be further illustrated by the following figures and examples. However, these examples and figures should not be interpreted in any way as limiting the scope of the present invention.

### FIGURES

FIG. 1: IDO activity controls gut microbiota-dependent regulation of obesity and its complications. (a-e) absence of IDO in non-myeloid compartment protects against obesity and insulin-resistance. % of weight gain (b), weights of ingWAT, epiWAT, retWAT and liver (c), insulin test tolerance (ITT) (d), oral glucose tolerance test (OGTT) (e) in WT mice irradiated and transplanted with either WT or Ido-1−/− bone marrow (Ido-1−/−->WT (n=10) and WT->WT (n=10) groups) or Ido-1−/− mice irradiated and transplanted with WT bone marrow (WT->Ido-1−/− (n=10)) after 20 weeks of HFD. (f) IDO activity (Kyn/Trp) in small intestines and colons of WT mice fed with either NCD (n=5) or HFD (n=4) and HFD-fed Ido-1−/− mice (n=4). (g-i) weight curves, (g and h) HOMA-IR index normalized to body weight of WT and Ido-1−/− mice either on antibiotic treatment (Ab) (n=10 per group) or WT and Ido-1−/− mice mixed in the same cages from 4 weeks of age (mix) (n=8 per group) or WT and Ido-1−/− mice untreated and separated in different cages (n=10 per group) (i). (j-n) gavage of WT mice with feces from 1MT-treated or not treated ob/ob mice (n=10 per group). Ratio of Kyn/Trp in feces of 1MT-treated or not treated ob/ob mice (n=4 per group) (j), body mass (k), weights of ingWAT, epiWAT, retWAT and liver (1), representative cytometry and quantification of M2-like macrophages (F4/80+CD11b+CD206+) in epiWAT (n=5 per group) (m) and HOMA-IR in WT mice which received feces from 1MT-treated or not ob/ob mice (n=10 per group) (n). Data are expressed as mean±sem. *P<0.05, **P<0.001, ***P<0.0001.

FIG. 2: IDO deficiency preserves the intestinal barrier through IL-22 in the setting of obesity. (a) PCA plot based on bacterial 16S rDNA gene sequence abundance in fecal content of WT and Ido-1−/− mice fed with either NCD or HFD. Axes correspond to principal components 1 (x-axis), 2 (y-axis) and 3 (z-axis). (b, c) bacterial-taxon-based analysis at the phylum level (b) and at family level (c) in the fecal microbiota. (d) IAA and Kyn levels in small intestines and colons of WT fed with either NCD (n=5) or HFD (n=4) and HFD-fed Ido-1−/− mice (n=4). (e) IL-17 and IL-22 contents in payer patches (PP) of WT and Ido-1−/− mice fed with either NCD or HFD (n=3-4 per group). (f) Reg3b and 3 g mRNA in intestines of HFD-fed WT and Ido-1−/− mice (n=3-4 per group). (g) SCFA contents in the fecal microbiota from HFD-fed WT and Ido-1−/− mice (n=9-10 per group). (h) Plasma LPS in WT and Ido-1−/− mice fed with either NCD or HFD (n=5 per group) after 20 weeks.

Data are expressed as mean±sem. *P<0.05, **P<0.001, ***P<0.0001.

FIG. 3: IAA decreases insulin resistance and adipose inflammation. (a) feces IAA (indole acetic acid) (b) ITT, and quantification of M2-like macrophages (F4/80+CD11b+CD206+ in epiWAT) and CD45+ cells (n=5 per group) (c-d), in WT mice supplemented or not with IAA (diluted in drinking water, 2 mg/ml) and put on HFD during 11 weeks (n=9-10 per group Data are expressed as mean±sem. *P≤0.05, **p<0.001, ***p<0.0001.

### EXAMPLE

Material & Methods

Mice.

Male C57Bl/6 Ido-1−/− mice were bought from the Jackson Laboratory (Jax) and bred in our facility. At weaning, mice were separated according to the genotype. Male ob/ob mice were bought from Janvier Laboratory at 4 weeks of age. Mice were fed with either a normal chow diet (NCD) (A03, SAFE, France) or subjected to diet-induced obesity containing 60% FAT (E15742-347, SSNIFF, Germany). High fat diet (HFD) was started at 7 weeks of age and continued for 20 weeks or less with ad libitum access to water and food. For chimerism experiment, we subjected 10 weeks old C57BL/6 WT and C57BL/6 Ido-1−/− to medullar aplasia by 9.5 gray lethal total body irradiation. We repopulated the mice with an intravenous injection of bone marrow cells isolated from femurs and tibias of male C57BL/6 WT and C57BL/6 Ido-1−/−. After 4 weeks of recovery, mice were fed a HFD for 20 weeks. In some experiments, IDO inhibitor (L-1methyl tryptophan, 1MT) (Sigma) was used at 2 mg/mL diluted in drinking water. We also subjected some mice to antibiotic treatment as described before1. All mice used in these experiments were bred and housed in a specific pathogen-free barrier facility. Experiments were conducted according to the French veterinary guidelines and those formulated by the European community for experimental animal use (L358-86/609EEC).

In Vivo Studies.

For oral glucose tolerance test (OGTT), mice were fasted overnight prior to an oral administration of 1-5 g/kg glucose. Blood was sampled from the tail vein at 0, 5, 15, 30, 60, 90 and 120 min in order to assay glucose concentration (OneTouch Ultra glucometer, LifeScan Europe). At 0, 15, 30, 60 min tail vein blood was collected, plasma samples were stored at −20° C. until they were analyzed for insulin concentration (Crystal Chem Inc., Downers Grove, USA). Insulin tolerance test (ITT) was performed in mice food deprived for 5 h prior to an intraperitonial injection of 1 U/kg insulin. Blood was sampled from the tail vein at 0, 5, 15, 30, 60 and 90 min in order to assay glucose concentration. Experiments with fecal gavage were done with fresh stool samples from either ob/ob control mice or ob/ob mice supplemented with 1MT during 6 weeks until 19 weeks. Briefly, stool were suspended in water and sieved through a 70 μm cell strainer (BD). These fecal suspensions were inoculated to C57Bl/6 WT mice via oral gavage with 400 μL of fecal suspension 4 times per week during 15 weeks of HFD.

Analysis of Metabolic Parameters.

Measurement of short chain fatty acids (SCFA) was performed as described previously 4 with slight modifications. A stock solution of SCFA metabolites (Sigma Aldrich, France) was prepared and serially diluted to get 10 calibration solutions. A working solution of internal standards (IS) was prepared in 0.15 M NaOH to get the following final concentrations: 75 mmol/L of D3-acetate, 3.8 mmol/L of D5-propionate, 2.5 mmol/L of 13C-butyrate, 0.5 mmol/L of D9-valerate (Sigma Aldrich). Stool samples were weighed (˜50 mg), dissolved in 200 μL of sodium hydroxide solution at 0.15 M (NaOH, Sigma Aldrich). Twenty microliters of the internal standard solution were added to stool samples and calibration solutions. Each sample was then acidified with 5 μL of hydroxide chloride 37% (Sigma Aldrich, France) and then extracted with 1.7 mL of diethyl ether (Biosolve, France). Samples were stirred gently for 1 hour and then centrifuged 2 min (5000 rpm, 4° C.). The organic layers were transferred into 1.5 ml glass vials and SCFAs were derivatized with 20 μL of tert-butyldimethylsilyl imidazole (Sigma Aldrich, France). Samples were incubated 30 min at 60° C. before analysis. Samples were finally analyzed by GC-MS (model 7890A-5975C, Agilent Technologies, France) using a 30 m×0.25 mm×0.25 μm capillary column (HP1-MS, Agilent Technologies, France). The temperature program started at 50° C. for 1 min, ramped to 90° C. at 5° C./min, then up to 300° C. at 70° C./min. Selected ion monitoring (SIM) mode was used to measure SCFA concentrations with ions at m/z 117 (acetate), 120 (D3-acetate), 131 (propionate), 136 (D5-propionate), 145 (butyrate and isobutyrate), 146 (13C-butyrate), 159 (valerate), 168 (D9-valerate).

Adipose Cell Isolation and Flow Cytometry Analyses.

The stromal vascular fraction (SVF) containing mononuclear cells and preadipocytes was extracted from adipose tissue. Adipose tissue from mice was digested using 10 mL digestion solution (7 mL Hank's Solution, 3 mL 7.5% BSA and 20 mg collagenase type II, Sigma). The digestion was performed at 37° C. using a shaker at 100 rpm for 20 min. After digestion, the adipocyte fraction (floating) was isolated and the solution containing the SVF was centrifuged at 1500 rpm at 4° C. for 5 min. The SVF pellet was resuspended in 1 mL fluorescence-activated cell sorter (FACS) buffer. After 15 min incubation with Fc Block (2.4G2, BD Biosciences), SVF cells were stained with appropriate antibodies conjugated to fluorochromes or isotype controls for 30 min at 4° C. in the dark: CD45 (30-F11), F4/80 (BM8), CD11b (M1/70), CMHII (M5/114.15.2) from eBiosciences, CD11c (HL3) from BD Biosciences and CD206 (C068C2) from Biolegend. Samples were acquired using an Fortessa cytometer (Becton Dickinson) and analyzed with FlowJo (TreeStar) software programs.

Adipose Tissue Culture.

Mouse adipose tissue biopsies (0.1 g) were minced and incubated in 1 mL of endothelial cell basal medium (PromoCell) containing 1% bovine serum albumin, penicillin (100 U/mL) and streptomycin (100 U/mL). Adipose tissue-conditioned medium (ATCM) were recovered after 24h and stored at −80° C. until analysis.

Cytokine Quantification.

Cytokine concentrations from ATCM were analyzed using ELISA kits. Adiponectin ELISA kit was from R&D Sytems. IL-17 and IL-22 were measured in PPs (Peyer's patches) extracts. Briefly, PPs were lysed in detergent buffer (RIPA) containing protease inhibitor (Roche). After centrifugation 13000 g-10 min at 4° C., protein quantification was performed on supernatants and then supernatants were stored at −20° until ELISA assay.

Quantitative Real Time PCR.

Macrophages and intestines were lysed in detergent buffer RLT and then subjected to RNA extraction and reverse transcription (Qiagen). Then, quantitative real-time PCR was performed on an ABI PRISM 7700 (Applied Biosystems) in triplicates.

Intestinal Content DNA Extraction

Fecal genomic DNA was extracted from the weighted stool samples using a method that was previously described 7, which is based on the European MetaHIT DNA extraction method.

16s rRNA Gene Sequencing

16s rDNA gene sequencing of fecal DNA samples was performed as previously described (Lamas et al, 2016). Briefly, the V3-V4 region was amplified and sequencing was done using an Illumina MiSeq platform (GenoScreen, Lille, France). Raw paired-end reads were subjected to the following process: (1) quality-filtering using the PRINSEQ-lite PERL script38 by truncating the bases from the 3′ end that did not exhibit a quality <30 based on the Phred algorithm; (2) paired-end read assembly using FLASH (fast length adjustment of short reads to improve genome assemblies)8 with a minimum overlap of 30 bases and a 97% overlap identity; and (3) searching and removing both forward and reverse primer sequences using CutAdapt, with no mismatches allowed in the primers sequences. Assembled sequences for which perfect forward and reverse primers were not found were eliminated. Sequencing data were analyzed using the quantitative insights into microbial ecology (QIIME 1.9.1) software package. The sequences were assigned to OTUs using the UCLUST algorithm9 with a 97% threshold of pairwise identity and classified taxonomically using the Greengenes reference database10. Rarefraction was performed (8,000 sequences per sample) and used to compare abundance of OTUs across samples. Biodiversity indexes were used to assess alpha diversity and α and β diversities were estimated using phylogenetic diversity and unweighted UniFrac. Principal component analyses (PCA) of The Bray Curtis distance with each sample colored according to phenotype were built and used to assess the variation between experimental groups. The. LDA effect size algorithm was used to identify taxa that are specific to experimental group11.

HPLC Quantifications Thawed stools from mice were extracted as previously described 12. L-tryptophan (Trp) and L-kynurenine (Kyn) were measured via HPLC using a coulometric electrode array (ESA Coultronics, ESA Laboratories, Chelsford, Mass., USA)13. Quantifications were performed by referencing calibration curves obtained with internal standards. Other compounds (IAA) were quantified via liquid chromatography coupled to mass spectrometry (LC-MS) by using a Waters ACQUITY ultraperformance liquid chromatography (UPLC) system equipped with a binary solvent delivery manager and sample manager (Waters Corporation, Milford, Mass., USA) and that was coupled to a tandem quadrupole-time-of-flight (Q-TOF) mass spectrometer equipped with an electrospray interface (Waters Corporation). Compounds were identified by comparing with the accurate mass and the retention time of reference standards in our in-house library, and the accurate masses of the compounds were obtained from web-based resources, such as the Human Metabolome Database (http://www.hmdb.ca) and the METLIN database (http://metlin.scripps.edu).

NanoString.

NanoString analysis was performed and analyzed according to the manufacturer's recommendations.

Statistical Analysis.

Values are expressed as means±s.e.m. The differences between groups were assessed using Student t-test or non-parametric Mann-Whitney test. Values were considered significant at P≤0.05. Differences corresponding to p<0.05 were considered significant. Statistical analysis was performed with GraphPad Prism (San Diego, Calif., USA).

Results

The inventors previously showed that obesity is associated with an increase of intestinal indoleamine 2-3 dioxygenase (IDO) activity, which shifts tryptophan (Trp) metabolism. They showed the beneficial effect of IDO invalidation on body weight and fat mass, insulin sensitivity and inflammation.

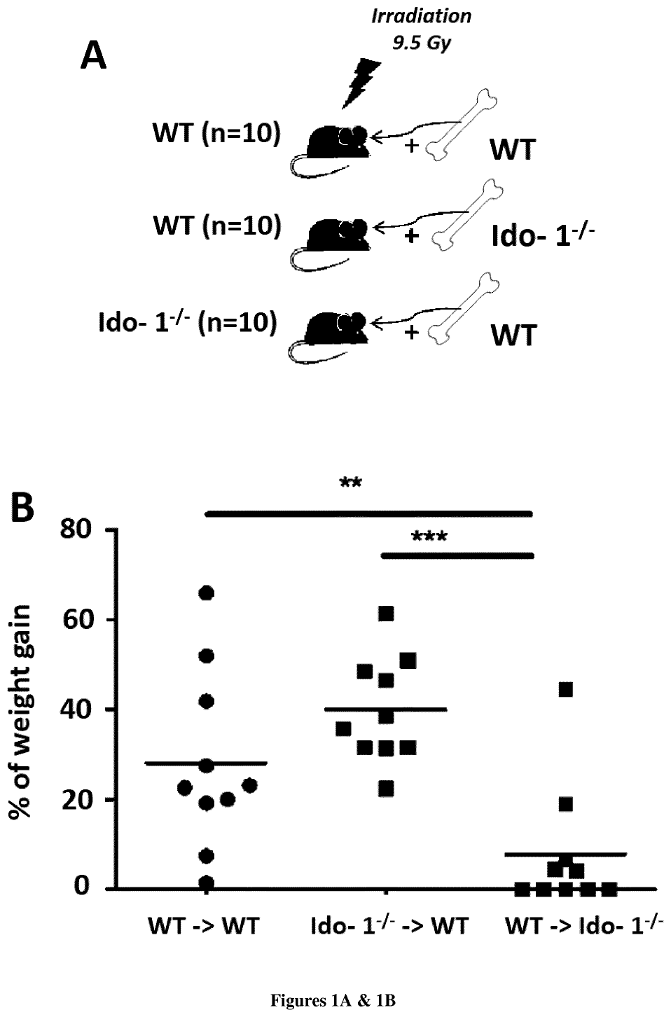

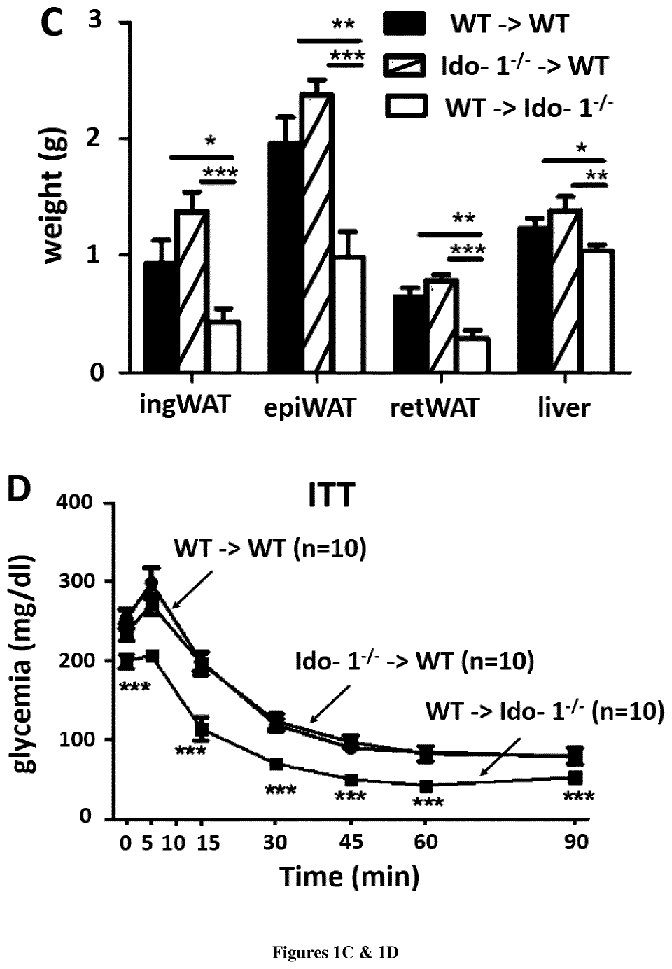

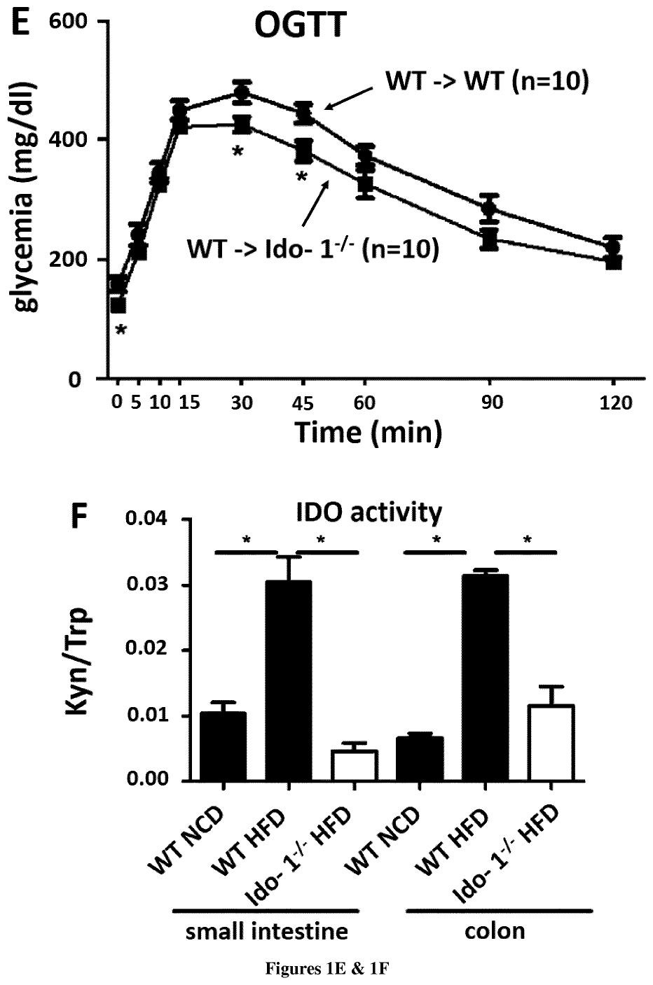

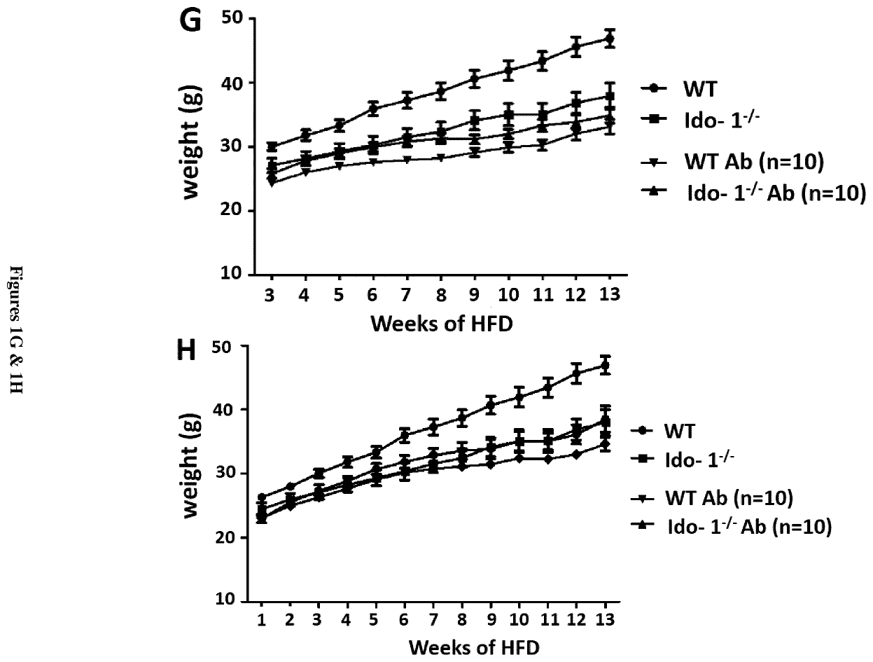

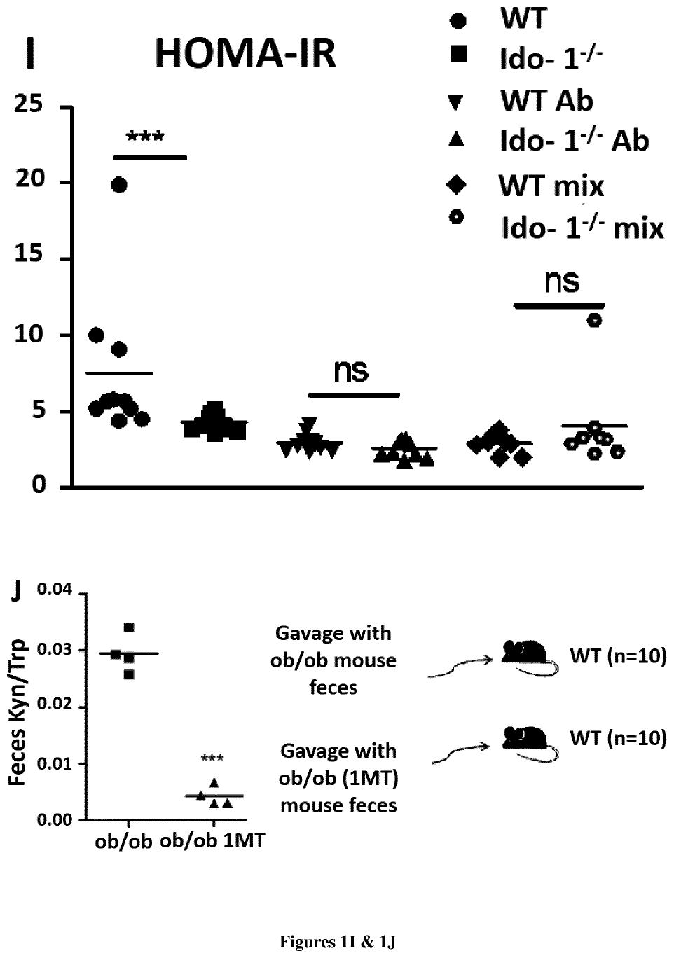

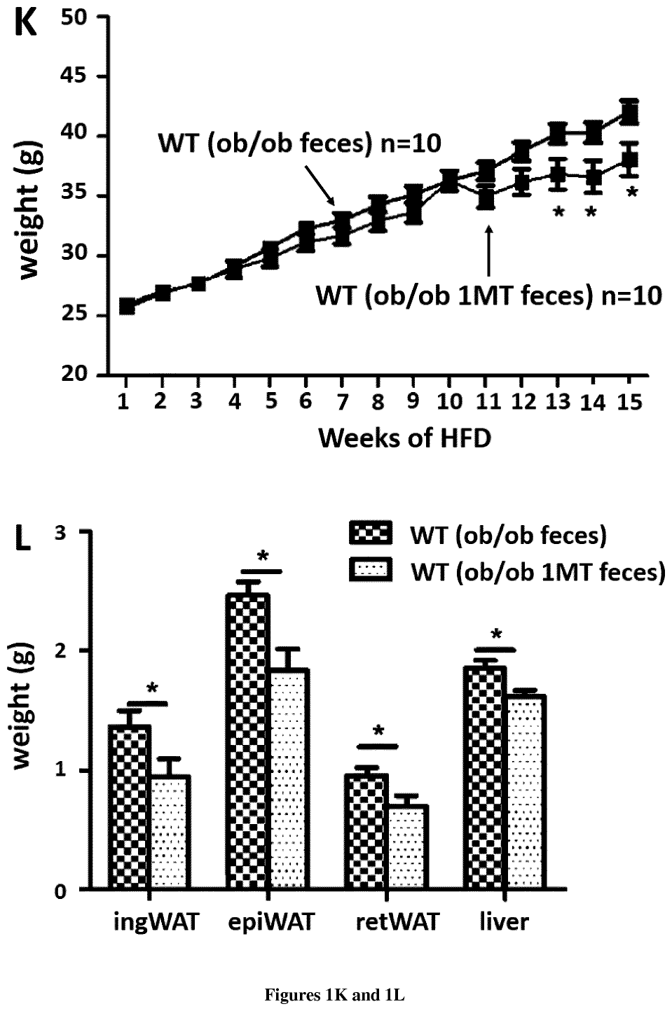

IDO is expressed by both myeloid and non-myeloid compartments. To distinguish between the roles of IDO in those compartments, we generated chimeric mice (FIG. 1a). Reconstitution of WT mice with bone marrow from Ido-1^{-/-} mice did not affect mouse body weight, WAT weights or insulin sensitivity (FIG. 1b-d). Interestingly, mice deficient for IDO in non-myeloid cells gained less body weight on HFD and had lower ingWAT, epiWAT, retWAT and liver weights (FIG. 1b-c), as well as improved insulin tolerance and glucose homeostasis (FIG. 1d-e), compared to HFD-fed WT mice transplanted with WT bone marrow, strongly supporting the importance of IDO expressed in non-myeloid compartment in the induction of metabolic disease.

Increased gut-derived lipopolysaccharide (LPS) translocation and intestinal dysbiosis were observed in obesity. Since IDO is expressed in the gastrointestinal tract, we analyzed intestinal IDO activity during HFD. As shown in FIG. 1f, HFD markedly increased IDO activity (Kyn/Trp) in both the small intestine and colon. We therefore hypothesised that intestinal IDO activity may hijack local Trp metabolism and shift it away from use by the gut microbiota.

To address the importance of the microbiota, we depleted the gut microbiota in WT and Ido-1^{-/-} mice using a broad spectrum antibiotic cocktail supplemented in drinking water. In agreement with a previous study, depletion of the microbiota protected the mice against HFD-induced gain weight (FIG. 1g). Moreover, antibiotic treatment abrogated the differences of body weight previously seen between HFD-fed WT and HFD-fed Ido-1^{-/-} mice (FIG. 1g). To test whether the gut microbiota is involved in the phenotype, WT and Ido-1^{-/-} mice were co-housed after weaning (mix) and compared to mice housed in cages separated by genotype. As shown in FIG. 1h, the weight of co-housed animals (whether WT or Ido-1^{-/-}) was similar to those of Ido-1^{-/-} mice housed in separate cages, indicating a dominant protective effect against weight gain of microbiota from Ido-1^{-/-} mice. Moreover, antibiotic treatment and co-housing abrogated the genotype-related differences in insulin-resistance index (HOMA-IR) (FIG. 1i).

We then sought to know whether microbiota transfer might suffice to recapitulate the phenotype observed in HFD-fed Ido-1^{-/-} mice. We thus forced-fed WT mice with feces collected from ob/ob mice treated or not with 1MT. We used ob/ob mice because they are already obese and they showed improved insulin sensitivity but no difference in body weight in response to 1MT treatment (data not shown), in association with a significant decrease of the ratio of Kyn/Trp in the feces (FIG. 1j). As shown in FIG. 1k-n, repetitive gavage of WT mice with feces from 1MT-treated ob/ob mice led to a lower increase of total body, WAT and liver weights, to a higher content of M2-like macrophages in epiWAT, and a lower insulin resistance index (HOMA-IR), compared to WT mice transferred with feces from control ob/ob mice, indicating protective effects of microbiota collected from mice treated with IDO inhibitor.

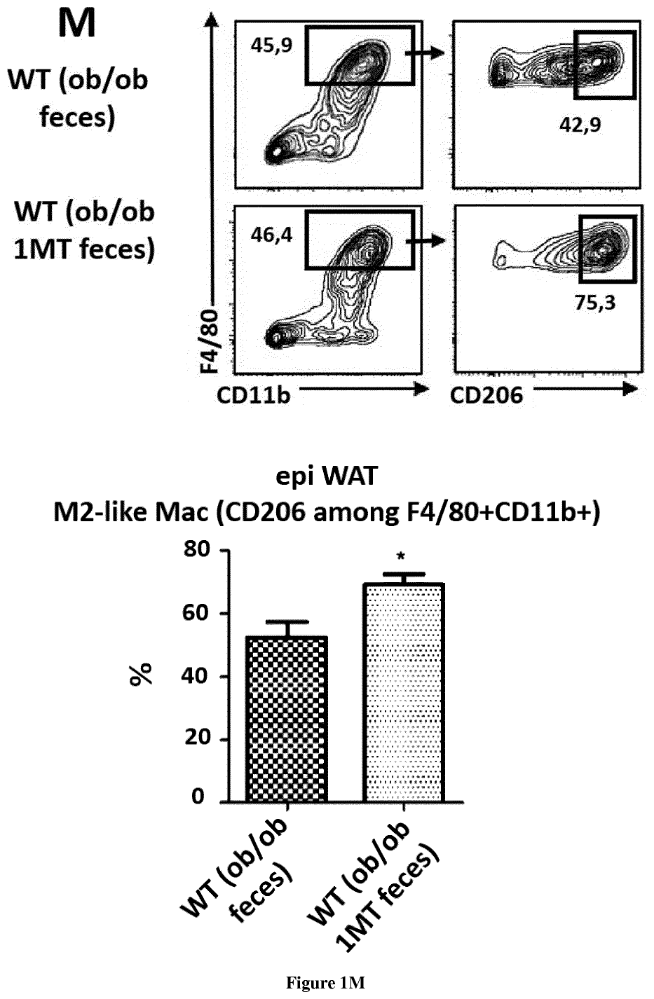

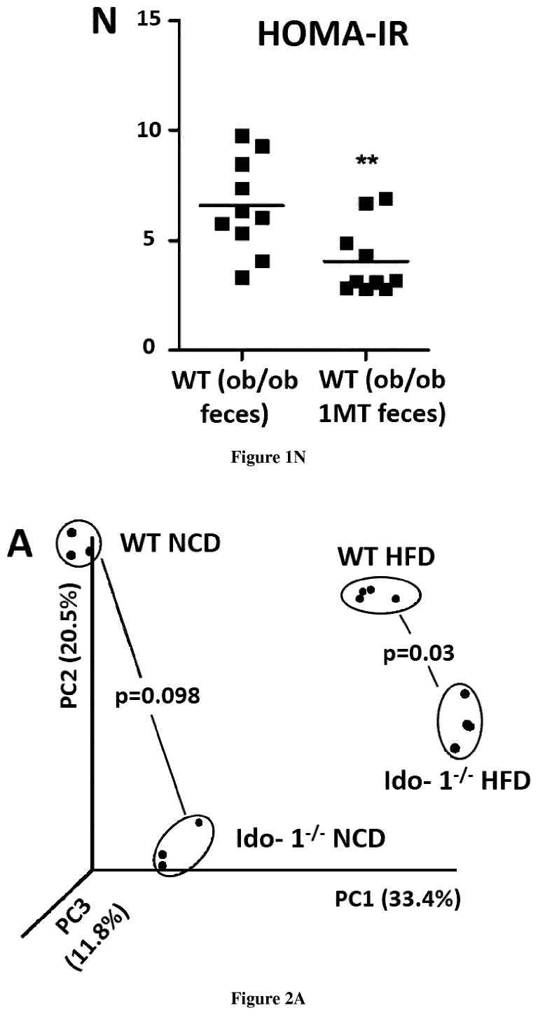

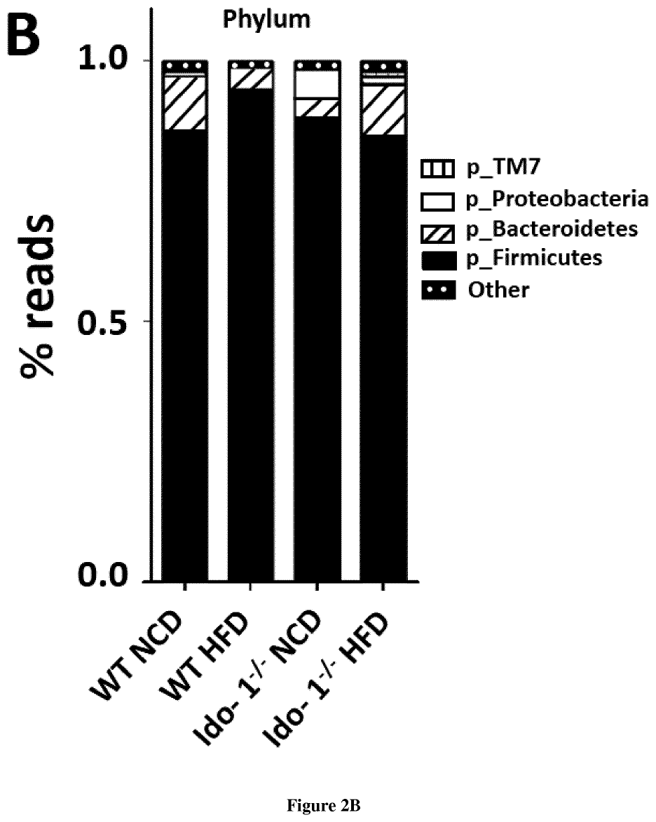

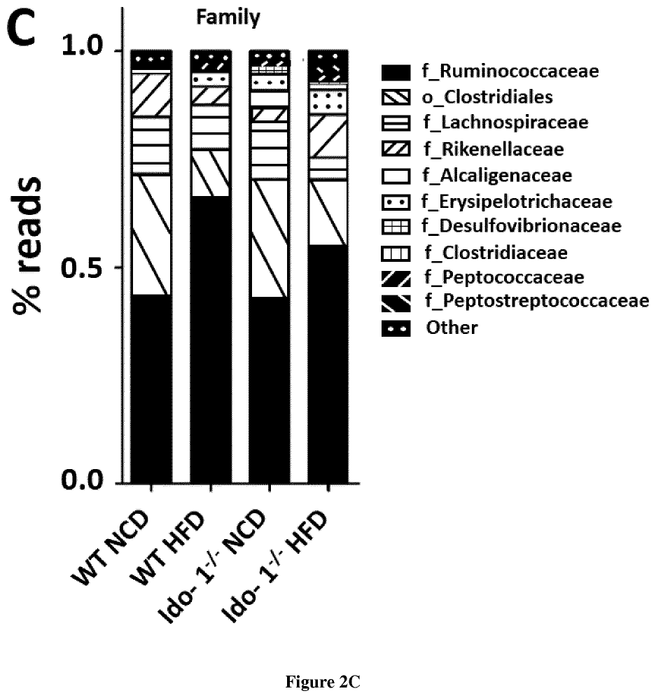

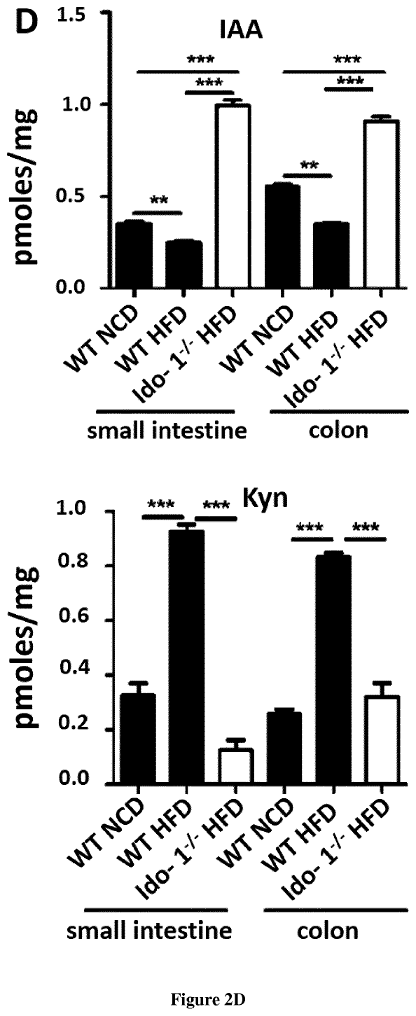

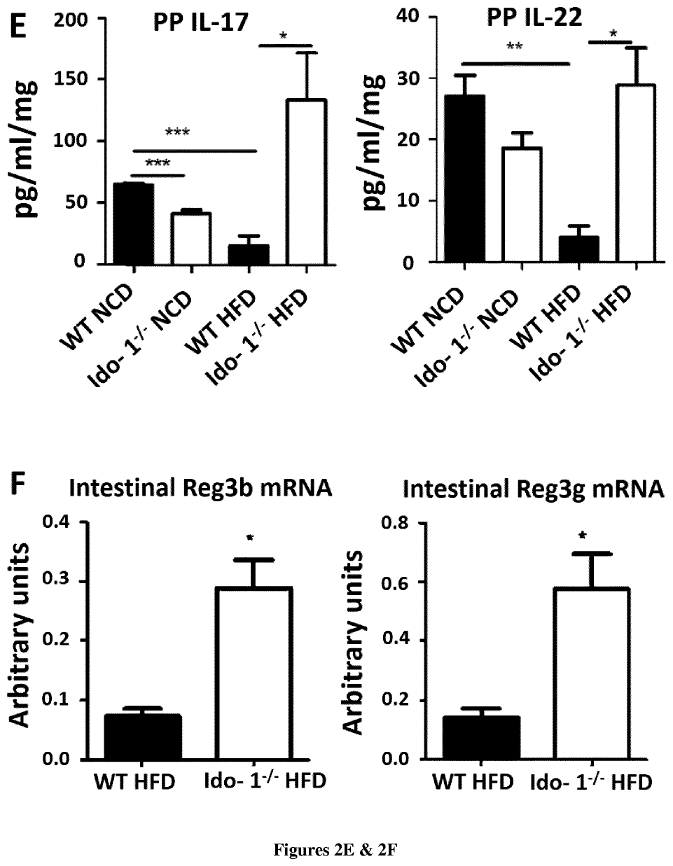

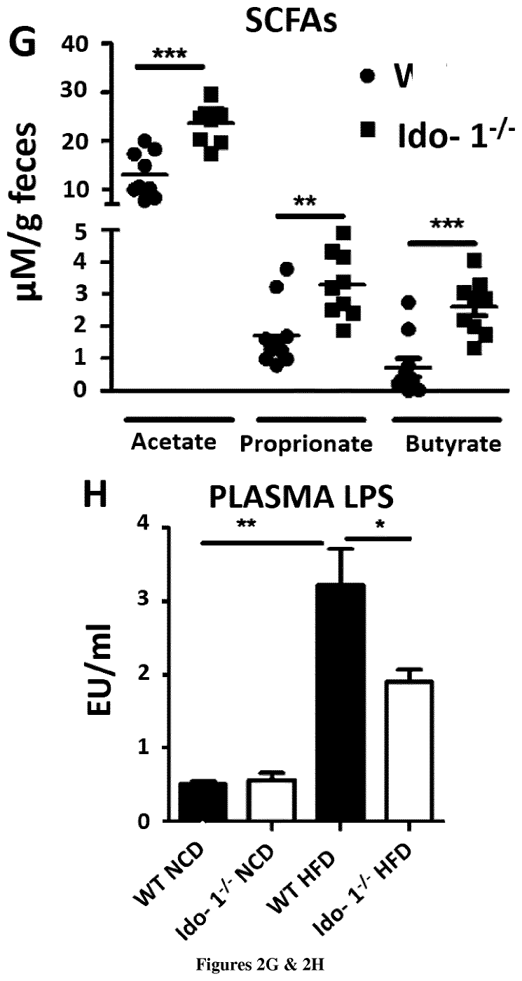

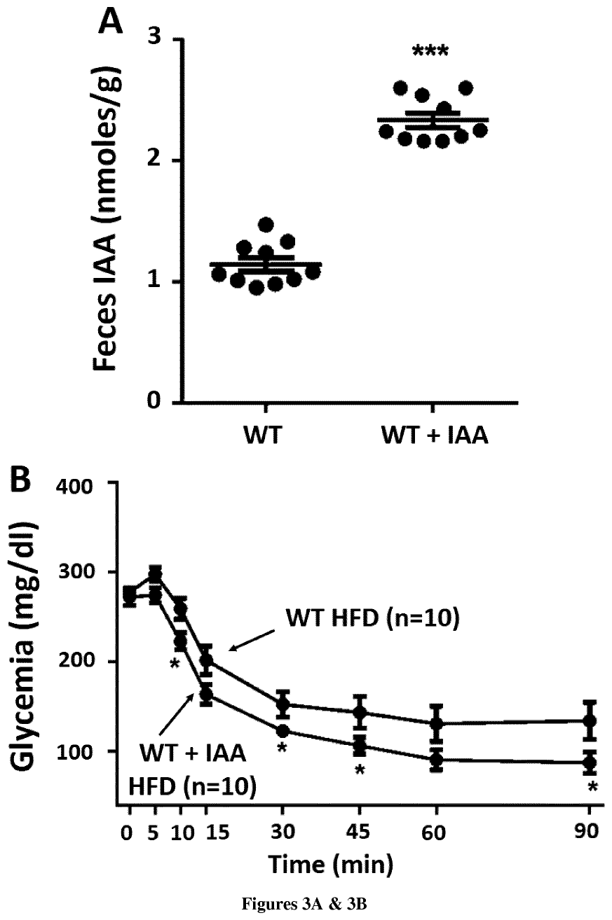

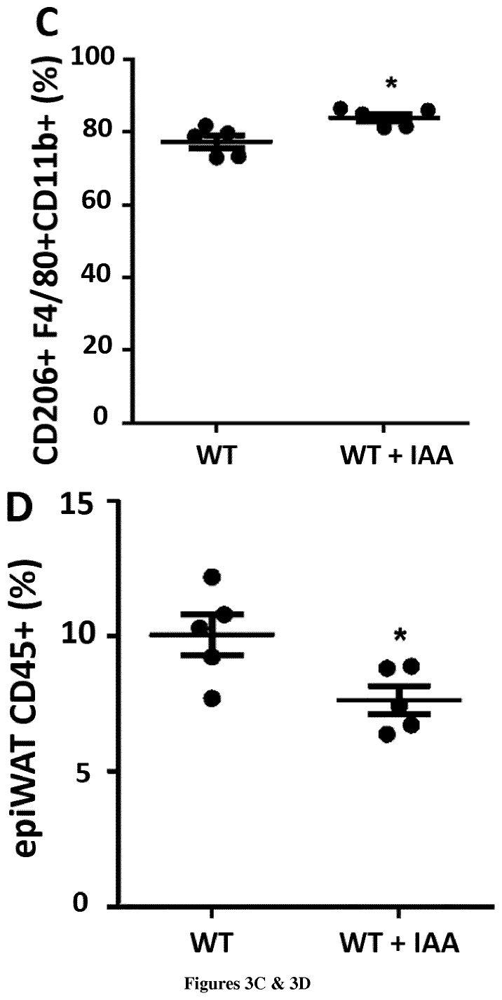

We next explored the bacterial fecal composition of the microbiota by use of 16S rDNA sequencing. Principal component analysis (PCA) on the basis of genus composition revealed major differences between WT and Ido-1^{-/-} mice fed with HFD (FIG. 2a). No differences regarding bacterial biodiversity were observed between WT and Ido-1^{-/-} mice fed with HFD (data not shown). At the phylum level, important differences were observed between WT and mice fed with either NCD or HFD (FIG. 2b). In particular, we found that the HFD increased the Firmicutes to Bacteroidetes ratio in WT mice, as previously reported, whereas Ido-1^{-/-} mice showed a reduction of this ratio (FIG. 2b). At the family level, significantly greater proportions of Ruminococcaeae and lower proportions of Rikenellaceae were observed in HFD-fed WT mice compared to NCD-fed WT mice (FIG. 2c), in agreement with previous reports. Whereas in HFD-fed Ido-1^{-/-} mice compared to NCD-fed Ido-1^{-/-} mice, the decrease of Firmicute was mainly due to a lower proportion of Clostridiales, in particular Lachnospiraceae (FIG. 2c). Overall, these data demonstrate that IDO has an important role in shaping gut microbiota, which is required to control body weight and insulin-resistance.

Trp is either metabolized by IDO to produce Kyn or by gut bacteria into indole derivatives, such as indole-3-acetic acid (IAA). We hypothesised that in obesity the increase of IDO activity shifts Trp metabolism from generation of indole derivatives towards Kyn production. To test this, we examined intestinal content of IAA, Trp and Kyn in NCD or HFD-fed WT or Ido-1^{-/-} mice. As shown in FIG. 2d, HFD decreased intestinal content of IAA, whereas it markedly increased Kyn levels in the gastrointestinal tract, indicating that HFD-induced obesity causes a major shift of Trp metabolism towards Kyn production. Consistently, in the case of a low level of intestinal Kyn as in HFD-fed Ido-1^{-/-} mice (FIG. 2d), a substantially higher IAA intestinal content was observed, as compared with HFD-fed WT mice (FIG. 2d) without any major changes of intestinal Trp levels (data not shown). This data supports the importance of IDO in controlling Kyn and IAA balance.

We then explored the role of the 2 cytokines related to indole metabolites, IL-17 and IL-22, in our findings. In agreement with previous reports showing that HFD decreased IL-17 and IL-22, we found lower levels of these cytokines in payer patches (PP) of HFD-fed WT compared to NCD-fed WT mice (FIG. 2e). Moreover, in accord with higher IAA, we observed more IL-17 and IL-22 in HFD-fed Ido-1^{-/-} mice compared to HFD-fed WT (FIG. 2e). Accordingly, we found an increase of IL-22-target genes such as antimicrobial proteins, regenerating islet-derived (Reg)3 g, Reg3b mRNA (FIG. 2f) in intestines of HFD-fed Ido-1^{-/-} compared to WT mice. Short-chain fatty acids (SCFAs), mainly acetate, propionate and butyrate, are the end products of fermentation of dietary fibers by the anaerobic intestinal microbiota, and have been shown to exert multiple beneficial effects. Interestingly, a higher fecal level of SCFAs was observed in HFD-fed Ido-1^{-/-} compared to WT mice (FIG. 2g) supporting a restoration of the intestinal ecosystem. As previously published, we found that plasma LPS increased with obesity (FIG. 2h). However, HFD-fed Ido-1^{-/-} mice showed lower plasma LPS in comparison to HFD-fed WT mice (FIG. 2h) Altogether these results provide a strong evidence for a protective role of IDO deletion in preserving intestinal immune barrier during obesity. IL-22 was shown to exert essential roles in eliciting antimicrobial immunity and maintaining mucosal barrier integrity within the intestine.

Altogether, our findings in mice provide strong evidence for a role of IDO in shifting Trp metabolism away from microbiota-dependent production of IL-22 and promotes obesity. This previously unknown function of IDO in fine tuning intestinal Trp metabolism makes IDO an attractive novel therapeutic target against metabolic diseases.

### REFERENCES

Throughout this application, various references describe the state of the art to which this invention pertains. The disclosures of these references are hereby incorporated by reference into the present disclosure.

1. A method of treating metabolic diseases, improving insulin sensitivity, controlling weight gain or stimulating weight loss in a subject in need thereof comprising administering to the subject a therapeutically effective amount of a probiotic.

2. The method of claim 1 wherein the metabolic disease is selected from the group consisting of diabetes, obesity, hypertension, elevated plasma insulin concentrations and insulin resistance, dyslipidemia, and hyperlipidemia.

3-4. (canceled)

5. The method according to claim 1, wherein the probiotic comprises Bacteroidetes.

6. The method according to claim 1, wherein the probiotic comprises Rikenellaceae.

7. The method according to claim 1, wherein the metabolic disease is obesity.

8. A composition comprising Bacteroidetes probiotics.

9. A composition comprising Rikenellaceae probiotics.

10. The method according to claim 1, wherein the probiotic is administered to the subject in the form of a dietary supplement or in the form of pharmaceutical composition.

11. A method of treating a metabolic disease, improving insulin sensitivity, controlling weight gain or stimulating weight loss in a subject in need thereof comprising administering to the subject a therapeutically effective amount of at least one ligand of aryl hydrocarbon receptor (AHR).

12-13. (canceled)
